(19) United States
(12) Patent Application Publication
(10) Pub. No.: US 2013/0131503 A1
(43) Pub. Date: May 23, 2013

(54) TRACKING A GUIDEWIRE

(71) Applicant: Ascension Technology Corporation, Sarasota, FL (US)

(72) Inventors: Mark Robert Schneider, Williston, VT (US); Jack Thomas Scully, Brewster, MA (US)

(73) Assignee: Ascension Technology Corporation, Sarasota, FL (US)

(21) Appl. No.: 13/683,703

(22) Filed: Nov. 21, 2012

Related U.S. Application Data
(60) Provisional application No. 61/562,991, filed on Nov. 22, 2011.

(51) Int. Cl. A61B 5/06

(52) U.S. Cl. CPC A61B 5/066
USPC 600/424

(57) ABSTRACT

In one aspect, in general, a method includes receiving, at a computer system, data from an electromagnetic sensor, determining, at the computer system, based on the received data, a location of a tip of a guidewire inserted in a patient, and causing, by the computer system, an indication of the determined location of the tip of the guidewire to be displayed in an overlay image representing at least part of the guidewire.

### PRIORITY CLAIM

This application claims the benefit of U.S. Provisional Application No. 61/562,991, filed Nov. 22, 2011, the contents of which are hereby incorporated by reference in their entirety.

### TECHNICAL FIELD

This disclosure relates to tracking a guidewire.

### BACKGROUND

Central venous access is an invasive procedure. Central venous access involves placing a long catheter that extends into the deep veins of the chest or abdomen. Central venous access provides a way to infuse agents that are caustic to the smaller veins of the arm. As a result, central venous access is used for chemotherapy, total parenteral nutrition, and numerous other agents. Larger diameter catheters are used for applications that require high flow rates such as hemodialysis, plasmaphersis, and volume resuscitation.

### SUMMARY

In one aspect, in general, a method includes receiving, at a computer system, data from an electromagnetic sensor, determining, at the computer system, based on the received data, a location of a tip of a guidewire inserted in a patient, and causing, by the computer system, an indication of the determined location of the tip of the guidewire to be displayed in an overlay image representing at least part of the guidewire.

Implementations of this aspect can include one or more of the following features. The overlay includes an x-ray image. The overlay includes an ultrasound image. The guidewire is inserted into a vein of the patient. Determining the location of a tip of a guidewire includes measuring three-dimensional coordinates of the guidewire. The method includes generating an x-ray image after the location of the tip of the guidewire has been determined. The tip of the guidewire includes an electromagnetic transmitter. The electromagnetic sensor is placed external to the patient.

In another aspect, in general, a method includes receiving, at a computer system, data from an electromagnetic sensor, determining, at the computer system, based on the received data, a location of a tip of a guidewire inserted in a patient, and providing, by the computer system, an indication to a user interface that the tip of the guidewire has been positioned at a predetermined location.

Implementations of this aspect can include one or more of the following features. The method includes determining, at the computer system, if a tip of a catheter has been positioned at the determined location of the tip of the guidewire, and providing, by the computer system to a user interface, an indication that the tip of the catheter has been positioned at the determined location of the tip of the guidewire. The predetermined location corresponds to a location of a target device. The target device is internal to the patient. The indication that the tip of the catheter has been positioned at the determined location comprises at least one of visual and audible confirmation.

In another aspect, in general, a system includes a transmitter of electromagnetic signals disposed on a tip of a guidewire, a sensor for receiving the electromagnetic signals transmitted by the sensor, a computer system in communication with the sensor, the computer system configured to determine a location of the tip of a guidewire based on the signals received by the sensor, and a display system in communication with the computer system, the display system configured to display an indication of the determined location of the tip of a guidewire in an overlay upon an image of at least part of the guidewire.

Implementations of this aspect can include one or more of the following features. The image includes an ultrasound image. The image includes an x-ray image. The computer system comprises an integrator for measuring rising edge and steady state of the electromagnetic signals. The transmitter comprises a multi-axis transmitter. The sensor comprises a one-axis coil. The transmitter provides pulsed DC current signals to each transmitter axis. The sensor comprises a 5 degrees-of-freedom sensor. The sensor comprises a pad that can be affixed to a patient.

In another aspect, in general, a computer program product is stored on a computer readable storage device, the computer program product including instructions that, when executed, cause a computer system to receive data from an electromagnetic sensor, determine, based on the received data, a location of a tip of a guidewire inserted in a patient, and cause an indication of the determined location of the tip of the guidewire to be displayed in an overlay upon an image representing at least part of the guidewire.

Implementations of this aspect can include one or more of the following features. The image includes an ultrasound image. The image includes an x-ray image.

These and other aspects and features and various combinations of them may be expressed as methods, apparatus, systems, means for performing functions, program products, and in other ways.

Other features and advantages will be apparent from the description and the claims.

### DESCRIPTION OF DRAWINGS

FIG. 1 shows a central venous catheter.

FIG. 2 is a block diagram of components of a guidewire tracking system.

FIG. 3 shows an electromagnetic sensor.

FIG. 4 shows a flowchart.

FIG. 5 shows anatomic landmarks.

FIG. 6 shows a flowchart.

FIG. 7 is a block diagram of a computer system.

Like reference symbols in the various drawings indicate like elements.

### DETAILED DESCRIPTION

A guidewire tracking system (GTS) that uses electromagnetic signals can allow a surgeon to visualize catheter placement continuously through a virtual image overlay (e.g., over an ultrasound image) while minimizing x-ray exposure to both the surgeon and the patient (e.g., a pediatric patient).

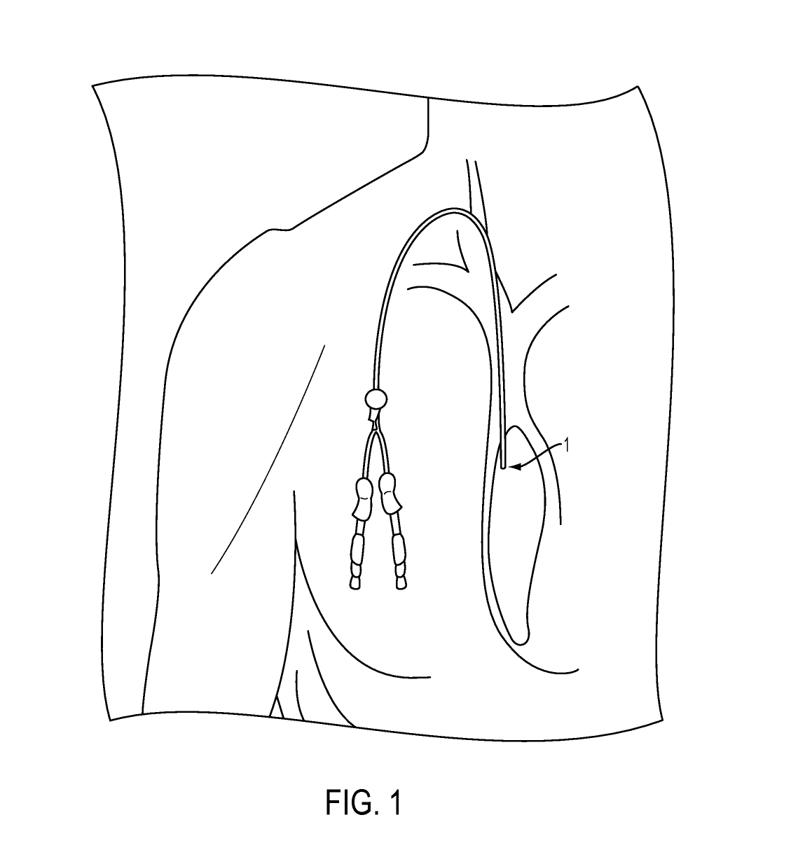

A guidewire is a device that is inserted into a patient undergoing a catheterization procedure and used to position a catheter. Central catheters, e.g., the central catheter shown in FIG. 1, can be placed in the operating room under general anesthesia using fluoroscopic guidance, which results in multiple x-ray images being used. Radiation may have negative side effects. The system described here can minimize or eliminate the use of radiation.

The system can also be adaptable for catheter placement in other settings, e.g., outside of the operating room, where catheters are inserted without the use of fluoroscopy. In this venue, catheter and guidewire manipulations are often done blindly. The lack of real-time feedback causes a variety of problems, which can lead to unsuccessful placement. For example, malpositioned catheters can lead to repeat procedures that in turn may increase the risk of infection, the potential for vascular injury, and the need for additional x-ray imaging for confirmation of placement.

Another procedure that can benefit from the system described herein is placement of a longer term intravenous line placed into a central vein in children. This procedure is used to give medicines, blood transfusions, fluids or nutrients. Blood tests may also be drawn through the catheter. The catheter is designed for long-time use so that many painful needle sticks can be avoided.

Imaging guidance can improve the success rate of catheter insertion by facilitating needle placement in the vein and catheter advancement to the target site. Ultrasound imaging is typically used to help guide the needle during initial access to the vein. The introduction of small, light, and cheap ultrasound units have facilitated compliance with this recommendation. However, ultrasound is not suitable for viewing the final placement of the catheter. For this purpose, fluoroscopy is used as described below.

Referring to FIG. 1, catheter placement is generally within a certain anatomical area, typically the superior vena cava above the right atrium 1, to avoid complications. Inserting a catheter too far increases the risks of cardiac arrhythmia and atrial perforation whereas not inserting the catheter far enough increases the risks of venous thrombosis and inadequate flow rates for dialysis and plasmapheresis.

Fluoroscopy is sometimes used during catheter insertion and the resulting feedback can increases the likelihood that the catheter tip will be positioned appropriately. An initial fluoroscopic image can be used to give an overall view and starting point, but subsequent fluoroscopy images can be avoided by real-time tracking of the guidewire tip using an electromagnetic sensor, and only one other final confirming fluoroscopy image may be required at the completion of the procedure, minimizing x-ray dose. In another example, neither an initial nor a final confirming fluoroscopy image is required. In other words, the operator can perform a procedure by relying only on the feedback from the electromagnetic sensor and the ultrasound. Guidewire tracking can be improved by using electromagnetic tracking technology. This technology is based on the generation of known electromagnetic field structures and couplings. Systems can be designed to measure 3 degrees-of-freedom (DOF), 5DOF and/or 6DOF. 3DOF typically corresponds to the 3 cardinal position coordinates, 5DOF to the 3 position and 2 orientation measurements (without roll) and 6DOF to the 3 position and 3 orientation (azimuth, elevation and roll) measurements. All systems utilize a source of electromagnetic fields. These can be AC, pulsed DC, permanent magnets, moving magnets, among others. There are also techniques for measuring the electromagnetic fields. This can be done with fluxgates, cored and non-cored coils that have induced voltages across them, Hall effect devices, magneto-resistors of all forms (e.g., plain, giant, colossal and tunneling), field dependent oscillators, squids, magnetometers, among others. These systems can operate in either direction, i.e., the tracked object can be generating or sensing a magnetic field, and the tracking system sensing or generating the magnetic field.

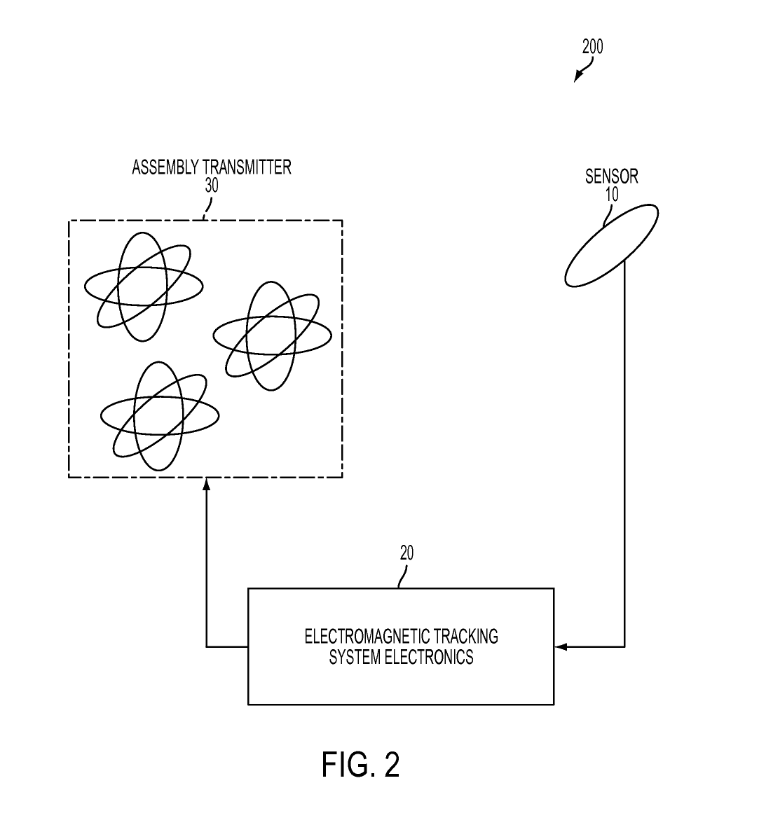

Referring to FIG. 2, in some implementations, a 5DOF pulsed DC tracking system 200 is employed for guidewire tracking. The electromagnetic tracking system electronics 20 consists of a computer component, a transmitter excitation component and a receiving component. Under computer command and control, a multi axis transmitter assembly 30 has each of its axes energized by DC drive electronics to transmit symmetrical, sequentially excited, nonoverlapping square DC-based waveforms. These are received through the air or tissue by one or more sensors 10 that conveys these signals to signal processing electronics within the electromagnetic tracking system electronics 20. The computer in the electromagnetic tracking system electronics 20 contains an integrator for measuring rising edge and steady state of each axes' sequential waveform so that an integrated result may be measured at the end of the steady state period. It further controls the transmitter DC drive electronics to operate the transmitter and receives signals from the signal processing electronics for the signal integration process, the end result being calculation of the sensor's position and orientation in three-dimensional space with significantly reduced eddy current distortion while providing improved compensation for sensor drift with respect to the Earth's stationary magnetic field and power-line induced noise.

Specifically, the transmitter DC drive electronics provides pulsed DC current signals of known amplitude to each transmitter axis. The computer sets the current amplitude for each transmitting element. The transmitter is configured to work near the patient undergoing the procedure. The one or more sensors 10 measures the position and orientation of the guidewire tip. The system is sufficiently versatile enough to accommodate other transmitter configurations and form factors depending on the medical procedure and the amount of conductive and ferrous metal in the nearby environment. In each case, the system computer is pre-programmed to accommodate the required configuration.

The one or more sensors 10 can each be a one-axis coil. The sensor is typically mounted in the distal tip of the guidewire that is guided or localized to an internal target within the patient or localized within the anatomy. The sensor detects pulsed DC magnetic fields generated by the transmitter and its outputs are conveyed to the signal processing electronics 30. The electronics control conditions and convert sensor signals into a digital form suitable for further processing by the computer and computation of position and orientation measurements.

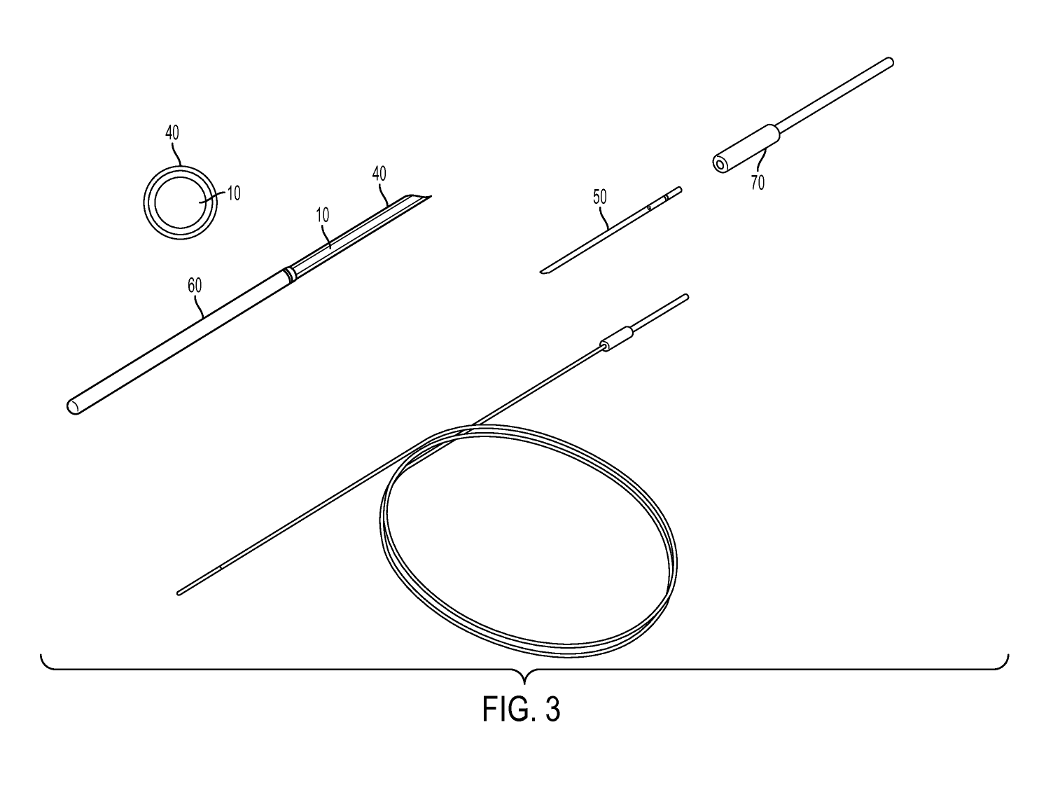

Referring to FIG. 3, a disposable 0.3 mm diameter 5DOF electromagnetic sensor 10 is placed near the end of a metallic braided wire tube 40 of roughly 50 cm in length. The metallic braided wire tube can preserve the flexibility during insertion and manipulation and has an approximately 0.85 mm outer diameter and inner diameter large enough to accommodate the sensor and sensor cables. The sensor 10 is sealed using an encapsulant, for example epoxy or some other medically acceptable material, to achieve applied part regulatory certification and make it impervious to blood or other bodily fluids. The metallic tube with sensor can be coated with PTFE (polytetrafluoroethylene) 50 to decrease and further protect the instrument. The overall outer diameter of the guidewire with coating will be 0.9 mm (0.035″), which allows a standard Broviac or Hickman catheter to be inserted over the guidewire.

A 20 mm long flexible Nitinol tip 60 with a 0.9 mm outer diameter can be positioned at the front of the guidewire to help minimize vessel trauma. The electrical wires of the electromagnetic sensor can be passed through the braided wire tube. At the far end from the sensor, a small connector can be included. This connector can be designed to be easily decoupled from the GTS connector 70. The connector can have insulated, concentric leads attached to the two sensor leads at the distal portion of the guidewire. This can mate with spring contacts contained within a cylindrical housing. This connector can allow, after positioning the guidewire in the patient's blood vessels, decoupling from the GTS to introduce the catheter along the guidewire.

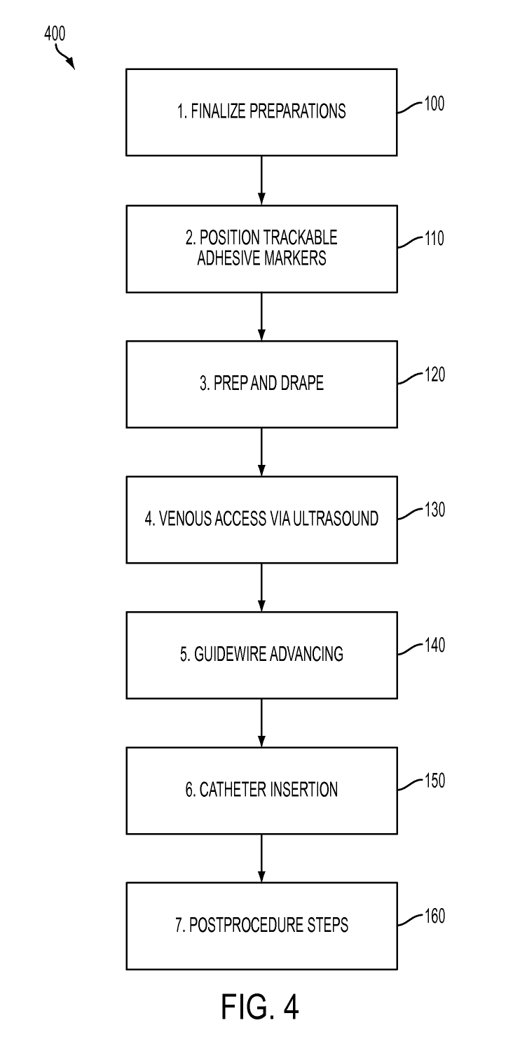

The GTS can provide visual information regarding the relative position and orientation of the guidewire. A flowchart 400 of the workflow is shown in FIG. 4. In block 100, the computer interface can require the operator to enter the planned procedure and indications for catheter placement. The interface can also prompt for compliance with standardized steps including informed consent, “time out”, site marking, and hand hygiene.

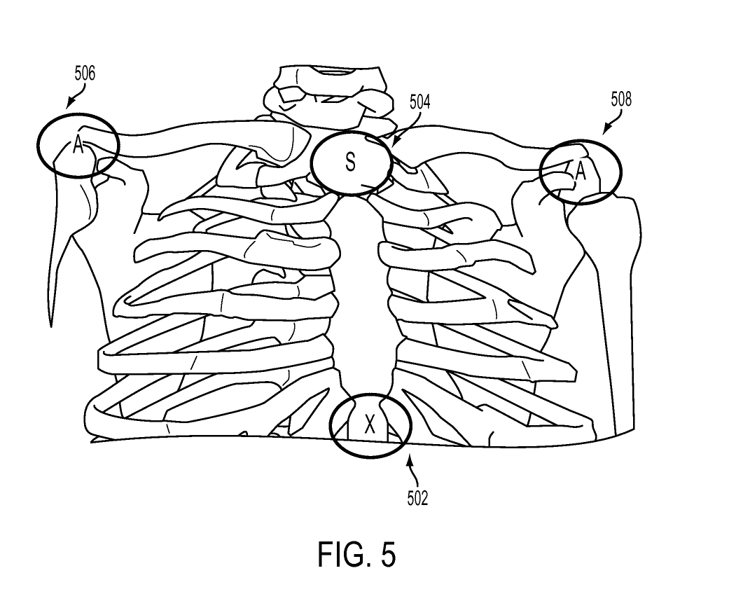

In block 110, the patient can be positioned on the table in the usual fashion. The GTS transmitter 30 (FIG. 1) can be placed near the patient and positioned to cover the workspace from the mid-neck to the diaphragm. Electromagnetically trackable pads can be fixed to external anatomic landmarks. These pads can consist of a single 5DOF sensor encapsulated onto a self-sticking pad. It is also possible to use 6DOF sensors. These landmarks can be used in system registration and to track patient movement. The anatomic landmarks can be the xiphoid 502, sternal notch 504, and both acromioclavicular joints 506, 508 as shown in FIG. 5, although others could be used depending on the procedure. This can allow referencing the guidewire position relative to these landmarks. Referencing is implemented to neutralize patient movement and respiration that might otherwise compromise accurate guidance of the guidewire to it anatomical destination.

Registration is accomplished by a number of techniques. Registration algorithms, based on touching multiple fiducial points in image space (reference frame #1) and patient space (reference frame #2), can be used for solving the registration problem. Some techniques for solving the registration problem involve directing the physician to place the tip of the instrument on fiducials, e.g., anatomical landmarks or markers affixed to the patient. In some examples, the trackable pads are placed on the anatomic landmarks before taking an x-ray, thereby capturing the locations of the pads in the x-ray. These data are then used in an algorithm, resident in the imaging software, to perform appropriate coordinate transformations and align image space to patient space, thus mapping the corresponding fiducials from one reference frame to another. A properly constructed registration algorithm accounts for shifts, rotations and scaling of points form one frame to another. The algorithm provides for a tight registration between frames with minimal errors between scanned images and targets. From this point on, the patient's anatomy is correlated to the image data. The imaging software can now display the position of the instrument's tip in the patient to its corresponding position in the image and vice versa. In many procedures, instruments are tracked on interactive displays, adjacent to the operational field or even displayed on a head-mounted display. Such displays allow the physician to see anatomy through a stereoscopic “window.” In this way, as an instrument's distal tip is moved toward an internal target, the physician can see a high-resolution, full-color stereoscopic rendering of the patient's anatomy and the trajectory to an internal target.

Block 120 indicates an operating procedure of prepping the vascular access site and ultrasound probe. In block 130, the operator can gain venous access using real-time ultrasound guidance. Guidewire tracking can start as the guidewire tip approaches the insertion site. The guidewire can then be inserted through a needle into a vein and the position of the guidewire can then be provided by the electromagnetic tracking system. Guidewire position and orientation can be displayed on a virtual image overlay using the original x-ray image. The user can then advance the guidewire in block 140 toward the target via guidance provided by the software and image display. In this example, the target location is the superior vena cava. When the tracked guidewire reaches the predetermined target, the system can provide visual and audible confirmation. In block 150, the catheter is then placed. The depth of the guidewire insertion before disconnecting the sensor cable can be noted. This measurement can be used to cut the catheter to the proper length. The catheter can then be placed over the guidewire. Finally, block 160 includes the steps of catheter securement, flushing, and radiograph and chart documentation.

In a second implementation, an x-ray is used at the start and end of the procedure to verify correct guidewire/catheter placement. In block 110, the patient can be positioned on the table in the usual fashion. Electromagnetically trackable pads can be fixed to external anatomic landmarks. These pads can consist of a single 5DOF sensor encapsulated onto a self-sticking pad along with a fiducial that can be visible in the x-ray image. It is also possible to use 6DOF sensors. The anatomic landmarks can be the xiphoid, sternal notch, and both acromioclavicular joints as shown in FIG. 5, although others could be used depending on the procedure. These landmarks can be used in system registration and to track patient movement. This can allow referencing the guidewire position relative to these landmarks. Referencing is implemented to neutralize patient movement and respiration that might otherwise compromise accurate guidance of the guidewire to it anatomical destination.

A portable x-ray unit can be brought into place and a single pre-procedure x-ray can be obtained. This x-ray may later be used to visualize the position of the tracked guidewire as described in block 150. The x-ray unit can be pulled back and the GTS transmitter 30 (FIG. 1) can then be placed near the patient and positioned to cover the workspace from the mid-neck to the diaphragm. Block 120 indicates standard operating procedure of prepping the vascular access site and ultrasound probe. Registration is accomplished as noted in the first implementation.

In block 130, the operator can gain venous access using real-time ultrasound guidance. Guidewire tracking can start as the guidewire tip approaches the insertion site. The guidewire can then be inserted through a needle into a vein and the position of the guidewire can then be provided by the electromagnetic tracking system. Guidewire position and orientation can be displayed on a virtual image overlay using the original x-ray image. The user can then advance the guidewire in block 140 toward the target via guidance provided by the software and image display. In this example, the target location is the superior vena cava. When the tracked guidewire reaches the predetermined target, the system can provide visual and audible confirmation. In block 150, the catheter is then placed. The depth of the guidewire insertion before disconnecting the sensor cable can be noted. This measurement can be used to cut the catheter to the proper length. The catheter can then be placed over the guidewire. Finally, block 160 includes the steps of catheter securement, flushing, and radiograph and chart documentation. A confirming x-ray can also be taken to validate the system performance and confirm final catheter placement.

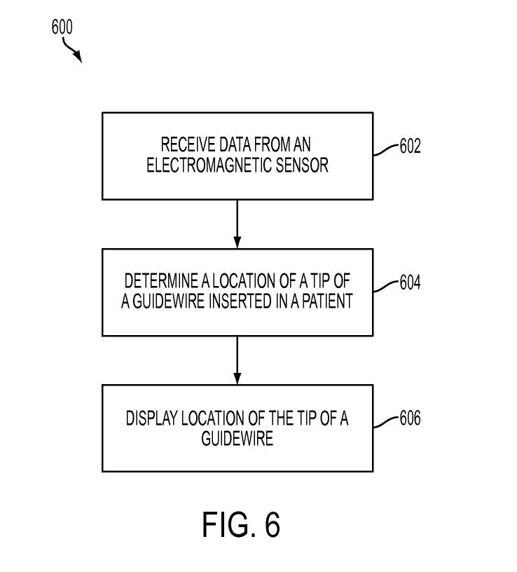

FIG. 6 shows a flowchart 600 of example operations of a guidewire tracking system. In step 602, data is received from an electromagnetic sensor. The sensor can be placed external to a patient undergoing a procedure. In some examples, the data is received from an electromagnetic transmitter disposed on the tip of a guidewire. In step 604, a location of a tip of a guidewire inserted in a patient is determined based on the received data. For example, a computer system can make the determination based on signals received from the sensor. In some examples, the guidewire is inserted into a vein of the patient. In some examples, three-dimensional coordinates of the guidewire are measured to determine the location of the tip. In some implementations, an x-ray image is generated after the location of the tip of the guidewire has been determined. In step 606, an indication of the determined location of the tip of the guidewire is caused to be displayed in an overlay upon an image, e.g., an ultrasound image, representing at least part of the guidewire. The indication could be visual, audible, or other type of signaling for confirmation, individually or in combination. In some examples, the ultrasound image is displayed in an overlay upon an x-ray image of the patient. In some examples, the overlay image is an x-ray image. In some examples, the system also indicates when a catheter, e.g., the tip of the catheter, has been positioned at a predetermined location, e.g., at the location of the tip of the guidewire.

Further, in some examples, a computer system provides an indication to a user interface that the tip of the guidewire has been positioned at a predetermined location. The predetermined location could correspond to a location of a target device (e.g., placed inside a patient).

FIG. 7 is a block diagram of an example computer system 700. For example, the guidewire tracking system can provide visual information regarding the relative position and orientation of the guidewire with the aid of a computer system 700. The computer system 700 includes a processor 710, a memory 720, a storage device 730, and an input/output device 740. Each of the components 710, 720, 730, and 740 can be interconnected, for example, using a system bus 750. The processor 710 is capable of processing instructions for execution within the system 700. In some implementations, the processor 710 is a single-threaded processor. In some implementations, the processor 710 is a multi-threaded processor. In some implementations, the processor 710 is a quantum computer. The processor 710 is capable of processing instructions stored in the memory 720 or on the storage device 730.

The memory 720 stores information within the system 700. In some implementations, the memory 720 is a computer-readable medium. In some implementations, the memory 720 is a volatile memory unit. In some implementations, the memory 720 is a non-volatile memory unit.

The storage device 730 is capable of providing mass storage for the system 700. In some implementations, the storage device 730 is a computer-readable medium. In various different implementations, the storage device 730 can include, for example, a hard disk device, an optical disk device, a solid-date drive, a flash drive, magnetic tape, or some other large capacity storage device. The input/output device 740 provides input/output operations for the system 700. In some implementations, the input/output device 740 can include one or more of a network interface devices, e.g., an Ethernet card, a serial communication device, e.g., an RS-232 port, and/or a wireless interface device, e.g., an 802.11 card, a 3G wireless modem, a 4G wireless modem, or another kind of interface. A network interface device allows the system 700 to communicate, for example, transmit and receive data over a network (e.g., the network 108 shown in FIG. 1). In some implementations, the input/output device can include driver devices configured to receive input data and send output data to other input/output devices, e.g., keyboard, printer and display devices 760. In some implementations, mobile computing devices, mobile communication devices, and other devices can be used. For example, the GTS can use a computer interface to allow the operator to enter the planned procedure and indications for the catheter placement. The computer interface could be an example of an input/output device 760. The GTS can also display visual information regarding the relative position and orientation of the guidewire on an input/output device 760. A server can be realized by instructions that upon execution cause one or more processing devices to carry out the processes and functions described above. Such instructions can comprise, for example, interpreted instructions such as script instructions, or executable code, or other instructions stored in a computer readable medium. A server can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

Although an example processing system has been described, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. Sometimes a server is a general purpose computer, and sometimes it is a custom-tailored special purpose electronic device, and sometimes it is a combination of these things.

Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

Certain features that are described that are described above in the context of separate implementations can also be implemented in combination in a single implementation. Conversely, features that are described in the context of a single implementation can be implemented in multiple implementations separately or in any sub-combinations.

The order in which operations are performed as described above can be altered. In certain circumstances, multitasking and parallel processing may be advantageous. The separation of system components in the implementations described above should not be understood as requiring such separation.

Other implementations not specifically described herein are also within the scope of the following claims.

What is claimed is:

1. A method comprising:
receiving, at a computer system, data from an electromagnetic sensor;
determining, at the computer system, based on the received data, a location of a tip of a guidewire inserted in a patient; and
causing, by the computer system, an indication of the determined location of the tip of the guidewire to be displayed in an overlay image representing at least part of the guidewire.

2. The method of claim 1, wherein the overlay includes an x-ray image.

3. The method of claim 1, wherein the overlay includes an ultrasound image.

4. The method of claim 1, wherein the guidewire is inserted into a vein of the patient.

5. The method of claim 1, wherein determining the location of a tip of a guidewire comprises measuring three-dimensional coordinates of the guidewire.

6. The method of claim 1 comprising generating an x-ray image after the location of the tip of the guidewire has been determined.

7. The method of claim 1, wherein the tip of the guidewire comprises an electromagnetic transmitter.

8. The method of claim 1, wherein the electromagnetic sensor is placed external to the patient.

9. A method comprising:
receiving, at a computer system, data from an electromagnetic sensor;
determining, at the computer system, based on the received data, a location of a tip of a guidewire inserted in a patient; and
providing, by the computer system, an indication to a user interface that the tip of the guidewire has been positioned at a predetermined location.

10. The method of claim 9 comprising
determining, at the computer system, if a tip of a catheter has been positioned at the determined location of the tip of the guidewire; and
providing, by the computer system to a user interface, an indication that the tip of the catheter has been positioned at the determined location of the tip of the guidewire.

11. The method of claim 10, wherein the predetermined location corresponds to a location of a target device.

12. The method of claim 11, wherein the target device is internal to the patient.

13. The method of claim 10, wherein the indication that the tip of the catheter has been positioned at the determined location comprises at least one of visual and audible confirmation.

14. A system, comprising
a transmitter of electromagnetic signals disposed on a tip of a guidewire;
a sensor for receiving the electromagnetic signals transmitted by the sensor;
a computer system in communication with the sensor, the computer system configured to determine a location of the tip of a guidewire based on the signals received by the sensor; and
a display system in communication with the computer system, the display system configured to display an indication of the determined location of the tip of a guidewire in an overlay upon an image of at least part of the guidewire.

15. The system of claim 14, wherein the image includes an ultrasound image.

16. The system of claim 14, wherein the image includes an x-ray image.

17. The system of claim 14, wherein the computer system comprises an integrator for measuring rising edge and steady state of the electromagnetic signals.

18. The system of claim 14, wherein the transmitter comprises a multi-axis transmitter.

19. The system of claim 14, wherein the sensor comprises a one-axis coil.

20. The system of claim 14, wherein the transmitter provides pulsed DC current signals to each transmitter axis.

21. The system of claim 14, wherein the sensor comprises a 5 degrees-of-freedom sensor.

22. The system of claim 14, wherein the sensor comprises a pad that can be affixed to a patient.

23. A computer program product stored on a computer readable storage device, the computer program product comprising instructions that, when executed, cause a computer system to:
receive data from an electromagnetic sensor;
determine, based on the received data, a location of a tip of a guidewire inserted in a patient; and
cause an indication of the determined location of the tip of the guidewire to be displayed in an overlay upon an image representing at least part of the guidewire.

24. The computer program product of claim 23, wherein the image includes an ultrasound image.

25. The computer program product of claim 23, wherein the image includes an x-ray image.
